(19) United States
(12) Patent Application Publication
(10) Pub. No.: US 2005/0149993 A1
(43) Pub. Date: Jul. 7, 2005

(54) Methods for treating circadian rhythm phase disturbances

(75) Inventors: Satchidananda Panda, San Diego, CA (US); John B. Hogenesch, Encinitas, CA (US); Ignacio Provincio, Arlington, VA (US); Steve A. Kay, San Diego, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignees: IRM LLC, Hamilton (BM); Uniformed Services University of the Health Sciences, Bethesda, MD (US); The Scripps Research Institute, La Jolla, CA (US)

(21) Appl. No.: 10/637,710

(22) Filed: Aug. 8, 2003

Related U.S. Application Data
(60) Provisional application No. 60/482,384, filed on Jun. 25, 2003.
(60) Provisional application No. 60/402,570, filed on Aug. 8, 2002.

(51) Int. Cl. A01K 67/027; C12N 5/06

(52) U.S. Cl. 800/3; 800/18; 435/354

(57) ABSTRACT

The present invention provides a transgenic non-human animals comprising a disruption in the melanopsin gene as well as methods for using the animals to identify agents useful for modulating circadian rhythm in animals.

### STATEMENT AS TO RIGHTS TO INVENTIONS MADE UNDER FEDERALLY SPONSORED RESEARCH AND DEVELOPMENT

This invention was made with Government support under Grant No. MH 62405, awarded by the National Institutes of Health. The Government has certain rights in this invention.

### CROSS-REFERENCE TO RELATED APPLICATIONS

This application claims benefit of U.S. provisional application No. 60/402,570, filed Aug. 8, 2002 and of U.S. provisional application No. 60/482,384, filed Jun. 25, 2003, each of which applications is herein incorporated by reference.

### BACKGROUND OF THE INVENTION

Most organisms coordinate their physiology and behavior in tune with the daily light cycle by utilizing a circadian clock that keeps track of the time of day. To be effective as a time-keeper, circadian clocks require endogenous oscillations to be stably entrained (synchronized) to the external environmental schedule. Thus, the external environmental schedule (e.g., the light/dark cycle) provides important temporal information.

A feature of this clock is that it entrains to perturbations in the light:dark cycle, such as during the changing of the seasons, thereby synchronizing the organism with changes in day length or light onset. Entrainment of the endogenous circadian oscillator to the external light/dark environment is achieved by light phase resetting of the oscillator. The phase adjustment induced by light depends on the phase of the cycle at which the light is perceived. In humans, light at the beginning of the “night” will delay the rhythm, whereas light administered toward the end of the night will advance the rhythm.

In mammals, photic entrainment is thought to involve retinal photoreceptors that signal via the retino-hypothalamic tract (RHT) to the master circadian oscillator residing in the suprachiasmatic nuclei (SCN) of the hypothalamus. Eenucleation of both eyes in nocturnal, as well as in diurnal rodents ablates light induced phase-shifts in behavioral rhythmicity. See, e.g., Nelson & Zucker., Comparative Biochemical Physiology 69A, 145-148 (1981)).

Although recent years have brought an increased understanding of the molecular mechanism of the circadian clock (reviewed in S. L. Harmer, et al., Annu Rev Cell Dev Biol 17:215-53 (2001)), identification of the photoreceptors that communicate light information to the clock has proven difficult (J. Bellingham, et al., Cell Tissue Res 309:57-71 (2002)). Several lines of evidence demonstrate that photoentrainment of circadian rhythms can occur in the absence of classical visual photoreceptors, rods and cones. For example, some visually blind human subjects and rod-less/cone-less mice can still reset their circadian oscillators in response to light (C. A. Czeisler, et al., N Engl J Med 332:6-11 (1995); M. S. Freedman, et al., Science 284:502-4 (1999)). Furthermore, an action spectrum of the resetting of the circadian clock to light peaks at 480 nm in retinally degenerate (rd) mice and at 500 nm in visually competent mice (T. Yoshimura, et al., J Comp Physiol 178:797-802 (1996)). While the 500 nm sensitivity suggests the involvement of rods, the 480 nm sensitivity differs significantly from the absorption spectra of rods and cones. Light may also indirectly entrain the oscillator in nocturnal animals by acutely suppressing activity (masking). Light masking of activity at intermediate to high irradiance levels helps to consolidate activity to the dark period (N. Mrosovsky, et al., J Comp Physiol 184:423-8 (1999)), which may ultimately entrain the oscillator. Taken in sum, these observations suggest mammals may have evolved to recruit an array of light input mechanisms to respond to daily changes in spectral composition and irradiance levels. In nature, however, the dim twilight period may play the most significant role in circadian light resetting in nocturnal animals, and initiating phase adjustments in diurnal animals (T. Roenneberg, R. G. Foster, Photochem Photobiol 66:549-61 (1997)). Therefore, photoreceptors that can integrate such low light intensity information, while at the same time exhibit a robust resistance to phase shifting by low intensity “photic noise” (moonlight) or short bright confounding stimuli (lightning) must play a significant role in circadian photoreception (J. S. Takahashi, et al., Nature 308:186-8 (1984); D. E. Nelson, et al., J Physiol 439:115-45 (1991)).

Disruption of circadian rhythms can result in a number of pathophysiological states in humans. The most common of these pathophysiological states is jet lag, though a number of other sleep or circadian rhythm disorders also occur. Therefore, there is a need in the art for methods of modulating circadian rhythms in mammals to treat these conditions. The present invention addresses this and other problems.

### BRIEF SUMMARY OF THE INVENTION

The present invention provides transgenic knockout mice whose genome comprises a disruption in the mouse's endogenous melanopsin gene. In some embodiments, the disruption prevents the expression of a functional melanopsin protein in cells of the mouse. In some embodiments, the mouse comprises a homozygous disruption of the melanopsin gene. In some embodiments, the homozygous disruption results in the transgenic knockout mouse exhibiting an attenuated circadian rhythm phase-shift in response to a light pulse during a dark portion of an environmental dark/light cycle.

The invention also provides cells isolated from the transgenic melanopsin knockout mouse described herein. In some embodiments, the genome of the cell comprises a disruption in its endogenous melanopsin gene, and the homozygous disruption prevents the expression of a functional melanopsin protein in said cell.

The invention also provides methods for identifying a therapeutic agent for modulating circadian rhythm in a mammal. In some embodiments, the methods comprise administering an agent to a transgenic knockout animal whose genome comprises a disruption in its endogenous melanopsin gene, wherein the disruption prevents the expression of a functional melanopsin protein in cells of the animal and the animal comprises a homozygous disruption of the melanopsin gene; and selecting an agent that modulates the regulation of circadian rhythm in the animal.

In some embodiments, the knockout animal displays an attenuated circadian rhythm phase-shift response to a light pulse during a dark portion of an environmental dark/light cycle.

In some embodiments, the selecting step comprises selecting an agent that enhances the animal's circadian rhythm phase-shift response to a light pulse during a dark portion of an environmental dark/light cycle. In some embodiments, the animal is a mouse.

The invention also provides methods of modulating circadian rhythm in a mammal in need thereof. In some embodiments, the method comprising administering to the mammal an effective amount of the agent selected by administering an agent to a transgenic knockout animal whose genome comprises a disruption in its endogenous melanopsin gene, wherein the disruption prevents the expression of a functional melanopsin protein in cells of the animal and the animal comprises a homozygous disruption of the melanopsin gene; and selecting an agent that modulates the regulation of circadian rhythm in the animal.

In some embodiments, timing of administration of the selected agent is pre-determined to coincide with an appropriate phase of an existing circadian rhythm to produce a selected modulation of the circadian rhythm. In some embodiments, the selected agent is used to treat or prevent a sleep disorder. In some embodiments, the mammal has a condition selected from the group selected from insomnia, Seasonal Affective Disorder, Shift Work dysrhythmia, delayed-sleep phase syndrome, and jet-lag. In some embodiments, the mammal is a human.

In some embodiments, the selected agent is administered in conjunction with melatonin or a compound that suppresses or stimulates melatonin production. In some embodiments, the selected agent is administered in conjunction with light therapy.

The present invention also provides methods of modulating circadian rhythm in a mammal in need thereof, the method comprising administering to the mammal an effective amount of a melanopsin modulator. In some embodiments, timing of administration of the modulator is pre-determined to coincide with an appropriate phase of an existing circadian rhythm to produce a selected modulation of the circadian rhythm. In some embodiments, the modulator is used to treat or prevent a sleep disorder.

In some embodiments, the mammal has a condition selected from the group selected from insomnia, Seasonal Affective Disorder, Shift Work dysrhythmia, delayed-sleep phase syndrome, and jet-lag. In some embodiments, the mammal is a human. In some embodiments, the modulator is administered in conjunction with melatonin or a compound that suppresses or stimulates melatonin production.

### Definitions

The term “gene” as used herein refers to a segment of DNA involved in producing a polypeptide chain. “Gene” includes regions preceding and following the coding region (leader and trailer) as well as intervening sequences (introns) between individual coding segments (exons).

The term “melanopsin gene” refers to a nucleic acid sequence encoding a polypeptide substantially identical to SEQ ID NO: 2 or SEQ ID NO:4. Exemplary melanopsin genes are capable of producing a transcript substantially identical to SEQ ID NO:1 or SEQ ID NO:3. Melanopsin genes can include the promoter, enhancer, and 5′ and 3′ untranslated regions that regulate transcription or translation of the melanopsin polypeptide.

“Melanopsin modulators” are used herein to refer to inhibitory or activating molecules of melanopsin expression or activity. Inhibitors are agents that, e.g., inhibit expression of a polypeptide of the invention or bind to, partially or totally block stimulation, decrease, prevent, delay activation, inactivate, desensitize, or down regulate the activity of a polypeptide of the invention, e.g., antagonists. Activators are agents that, e.g., induce or activate the expression of a polypeptide of the invention or bind to, stimulate, increase, open, activate, facilitate, or enhance activation, sensitize or up regulate the activity of a polypeptide of the invention, e.g., agonists. Modulators include naturally occurring and synthetic ligands, antagonists, agonists, small chemical molecules and the like. Assays for inhibitors and activators include, e.g., applying putative modulator compounds to cells expressing a polypeptide of the invention and then determining the functional effects on a polypeptide of the invention activity. Samples or assays comprising a polypeptide of the invention that are treated with a potential modulator are compared to control samples without the modulator to examine the extent of effect. Control samples (not treated with modulators) are assigned a relative activity value of 100%. Inhibition of a polypeptide of the invention is achieved when the polypeptide activity value relative to the control is less than about 80%, optionally 50% or 25, 10%, 5% or 1%. Activation of the polypeptide is achieved when the polypeptide activity value relative to the control is at least 110%, optionally 150%, optionally 200, 300%, 400%, 500%, or 1000-3000% or more higher.

A “circadian rhythm” refers to an internal daily biological clock in an organism. Typically circadian rhythms oscillate with an approximate 24 hour periodicity.

A “circadian rhythm phase shift” refers to a change in the phase of locomotor of an animal, typically in response to a perturbation in the animal's internal clock. When a perturbation is applied to an animal, it is common to observe that the time at which an event occurs (i.e. the phase) is often different than a control that did not receive the perturbation. This phase shift is usually measured in hours or minutes from the control (or in degrees from a 360° cycle or in circadian time). The magnitude of the phase shift usually depends on the time in the cycle at which the perturbation was applied. The phase shift of two animals can be compared by providing the same perturbation to the light/dark cycle of the animals and then measuring a change in phase shift. Phase shift of the first animal (e.g., a melanopsin knockout animal) is attenuated compared with the phase shift of second animal (e.g., a wild type or other control animal) if the phase shift observed for the first animal is less than the phase shift observed for the second animal. Phase shift can be measured as a percentage of the control. Exemplary attenuated phase shifts can be at least 5%, 10%, 15%, 20%, 25%, 30%, 35%, 40%, 45%, 50%, 60%, 70%, 80%, 90% or 95% of the control.

A “disruption that prevents expression of a gene” refers to any alteration (insertion, deletion, point mutation) that prevents translation of the encoded polypeptide or prevents transcription of an RNA encoding the polypeptide.

“Light therapy” refers to exposure of a subject to light with the goal of supplementing the amount of light a subject normally receives. Light therapy is can be used to treat such disorders and SADS, which is caused by long, dark winters.

The term “nucleic acid” or “polynucleotide” refers to deoxyribonucleotides or ribonucleotides and polymers thereof in either single- or double-stranded form. Unless specifically limited, the term encompasses nucleic acids containing known analogues of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions) and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); and Cassol et al. (1992); Rossolini et al., Mol. Cell. Probes 8:91-98 (1994)). The term nucleic acid is used interchangeably with gene, cDNA, and mRNA encoded by a gene.

The terms “polypeptide,” “peptide” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymers. As used herein, the terms encompass amino acid chains of any length, including full-length proteins (i.e., antigens), wherein the amino acid residues are linked by covalent peptide bonds.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine.

Amino acids may be referred to herein by either the commonly known three letter symbols or by the one-letter symbols recommended by the IUPAC-IUB Biochemical Nomenclature Commission. Nucleotides, likewise, may be referred to by their commonly accepted single-letter codes.

The terms “identical” or percent “identity,” in the context of two or more nucleic acids or polypeptide sequences, refer to two or more sequences or subsequences that are the same sequences. Two sequences are substantially identical if the two sequences have a specified percentage of amino acid residues or nucleotides that are the same (i.e., 60% identity, optionally 65%, 70%, 75%, 80%, 85%, 90%, or 95% identity over a specified region, or, when not specified, over the entire sequence), when compared and aligned for maximum correspondence over a comparison window, or designated region as measured using one of the following sequence comparison algorithms or by manual alignment and visual inspection. The invention provides polypeptides or polynucleotides that are substantially identical to the polypeptides or polynucleotides, respectively, exemplified herein (e.g., SEQ ID NO:1 or SEQ ID NO:3; or SEQ ID NO:2 or SEQ ID NO:4). This definition also refers to the complement of a test sequence. Optionally, the identity exists over a region that is at least about 50 nucleotides or amino acids in length, or more preferably over a region that is 100 to 500 or 1000 or more nucleotides or amino acids in length.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters.

A “comparison window”, as used herein, includes reference to a segment of any one of the number of contiguous positions selected from the group consisting of from 20 to 600, usually about 50 to about 200, more usually about 100 to about 150 in which a sequence may be compared to a reference sequence of the same number of contiguous positions after the two sequences are optimally aligned. Methods of alignment of sequences for comparison are well known in the art. Optimal alignment of sequences for comparison can be conducted, e.g., by the local homology algorithm of Smith and Waterman (1970) Adv. Appl. Math. 2:482c, by the homology alignment algorithm of Needleman and Wunsch (1970) J. Mol. Biol. 48:443, by the search for similarity method of Pearson and Lipman (1988) Proc. Nat'l. Acad. Sci. USA 85:2444, by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Ausubel et al., Current Protocols in Molecular Biology (1995 supplement)).

Two examples of algorithms that are suitable for determining percent sequence identity and sequence similarity are the BLAST and BLAST 2.0 algorithms, which are described in Altschul et al. (1977) Nuc. Acids Res. 25:3389-3402, and Altschul et al. (1990) J. Mol. Biol. 215:403-410, respectively. Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) or 10, M=5, N=−4 and a comparison of both strands. For amino acid sequences, the BLASTP program uses as defaults a wordlength of 3, and expectation (E) of 10, and the BLOSUM62 scoring matrix (see Henikoff and Henikoff (1989) Proc. Natl. Acad. Sci. USA 89:10915) alignments (B) of 50, expectation (E) of 10, M=5, N=−4, and a comparison of both strands.

The BLAST algorithm also performs a statistical analysis of the similarity between two sequences (see, e.g., Karlin and Altschul (1993) Proc. Natl. Acad. Sci. USA 90:5873-5787). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001.

An indication that two nucleic acid sequences or polypeptides are substantially identical is that the polypeptide encoded by the first nucleic acid is immunologically cross reactive with the antibodies raised against the polypeptide encoded by the second nucleic acid, as described below. Thus, a polypeptide is typically substantially identical to a second polypeptide, for example, where the two peptides differ only by conservative substitutions. Another indication that two nucleic acid sequences are substantially identical is that the two molecules or their complements hybridize to each other under stringent conditions, as described below. Yet another indication that two nucleic acid sequences are substantially identical is that the same primers can be used to amplify the sequence.

### BRIEF DESCRIPTION OF THE DRAWINGS

FIG. 1 illustrates the constructs used to generate the melanopsin Opn4^{-/-} knockout mouse. In FIG. 1A, the 5′UTR (white box) and exon 1 (dark box) of Opn4 gene was replaced with a neomycin gene. A 3.1 kb genomic DNA harboring the 5′ upstream region and a 3.2 kb genomic fragment harboring exons 2 through 6 mediated the homologous recombination (dashed lines). FIG. 1B illustrates how the mice were genotyped by PCR amplification using primer pairs ac or df (null allele), and bc or ef (wildtype allele).

FIG. 2 demonstrates that Opn4^{-/-} mice exhibit normal circadian behavioral rhythms and light suppression of activity. Littermate Opn4^{-/-}, Opn4−/+ and Opn4+/+ mice were entrained to a light:dark cycle, then allowed to free run under constant darkness. The locomotor activity data was collected and analyzed as described herein. The respective period lengths (mean±SD) under constant darkness were 23.7+0.14 (Opn4^{+/+}), 23.59+0.14 (Opn4^{+/-}), and 23.71+0.12 hr (Opn4^{-/-}) with no significant difference among genotypes (two-tailed, equal variance t-test, n=6-12 per genotype group). Representative activity traces of (A) Opn4^{+/+} and (B) Opn4^{-/-} mice are shown. Activity traces from the last four days of entrainment (LD) and 15 days of constant darkness (DD) are shown. Each horizontal line represents data from a single day; activity bursts of 1 minute bin along the horizontal axis. Normal light suppression of activity in the (C) Opn4^{+/+} and (D) Opn4^{-/-} mice. A 300 lux white light pulse was administered during the dark phase of entrainment. The light pulse acutely suppressed activity compared to activity at similar phase in the preceding or subsequent days. Representative records from 9 WT Opn4^{+/+} and 11 Opn4^{-/-} are shown. The light phase is indicated by a white background and the dark phase is indicated by a grey background. The light pulse was administered on day 6 at 2 hr after lights off.

FIG. 3 illustrates the attenuated photoentrainment in Opn4^{-/-} mice. A single 15 minute pulse of monochromatic light of 480 nm (blue star) was administered at 3 hours after activity onset, and produced a smaller phase shift in activity rhythm in the Opn4^{-/-} animal (A), compared to that in the wildtype littermate (B). The phase shift (red bar) on the day after the light pulse was determined by the Clocklab software package (Actimetrics, Evanston, Ill.). The light induced phase shift defect in the null mice is more pronounced at lower irradiance than at saturating light levels (C). Mean (±SEM) of phase shift measurements for the Opn4^{-/-} mice (black) and the littermate wildtype mice (grey) are shown (n=5-9 mice per group). Data was analyzed by t-test (two tailed, equal variance), and the significant difference (p<0.005) between genotypes is highlighted by an asterisk.

FIG. 4 illustrates entrainment deficiency in Opn4^{-/-}; rd/rd mice. Representative double plotted wheel running activity records of mice during photoentrainment and free-run in DD. WT (a), Opn4^{-/-} (b), and rd/rd (c) mice entrained to light dark cycle of 8 hours of 100 lux white light and 16 hours of darkness (LD::8:16), while Opn4^{-/-}; rd/rd (d) mice did not entrain. The slope of activity onset is shown with a solid line. Slope of activity onset of four additional Opn4^{-/-}; rd/rd mice are shown with broken or grey lines. After two weeks of constant darkness, three Opn4^{-/-}; rd/rd mice were again subjected to entraining conditions of LD8:16 with 800 lux white light. Local time is indicated at the top, while the light and dark periods are indicated by white and grey backgrounds, respectively.

FIG. 5 illustrates wheel running activity records of Opn4^{-/-}; rd/rd mice under different photoperiods. Representative double plotted activity records of WT (a) and O Opn4^{-/-}; rd/rd (b) mice under entraining conditions of 12 hr white light (800 lux) and 12 hr darkness (grey box). While the WT mice consolidate their activity to the dark phase and the time of activity onset is coincident with the light to dark transition, the Opn4^{-/-}; rd/rd mice continue to free run with an intrinsic period length of <24 hr. Representative double-plotted activity records of WT (c), Opn4^{-/-} (d), rd/rd (e), and Opn4^{-/-}; rd/rd (f) mice subjected to an entraining LD regime (8:16; 100 lux L) for 10 days followed by constant light (100 lux) for 20 days. While the LL regime extends the free running period of the oscillator to >24 hr in WT, Opn4^{-/-}, and rd/rd mice, it has no period lengthening effect in Opn4^{-/-}; rd/rd mice.

FIG. 6 illustrates acute suppression of locomotor activity by a 2-hour pulse of white light during the early night. The activity suppression is estimated by comparing the percent of daily activity during the time of light pulse (target activity) with average percent daily activity during comparable time over three previous nights (control activity), and is calculated as 100×[(control activity-target activity)/control activity]. Values from individual animals (diamonds) as well as group average values (horizontal bars) are shown.

FIG. 7 shows that a modifier locus on mouse chromosome 17 modulates acute light suppression of activity (masking) in Opn4^{-/-} mice. In a 129S1/SvImJ;C57BL/6 mixed genetic background, Opn4^{-/-} mice exhibit wide variation in masking, which is quite apparent when the imposed entraining LD cycle is phase-delayed. Representative double plotted wheel running activity records of WT (a) and Opn4^{-/-} (b) mice showing suppressed activity, and of some Opn4^{-/-} mice (c) showing no activity suppression during the light phase of the first few days of re-entrainment to the new LD cycle. Genotypes of sixteen Opn4^{-/-} mice showing no activity suppression by light were determined at 500 loci polymorphic between 129S1/SvlmJ and C57BL/6 strains (S2). A 6 Mb region on chromosome 17 was found to be overrepresented in these mice. The genotype of each of the 16 mice over this chromosomal region (d), and the summary of the genome scan (e) are shown.

FIG. 8 illustrates irradiance-response curves for pupillary constriction after exposure to 30 seconds of monochromatic 470 nm light. The percent pupillary constriction is calculated as 100×(1−(minimum pupil area during the 30 sec light pulse/dark-adapted pupil area)) (Mean±SEM is displayed).

FIG. 9 illustrates photoinhibition of the nocturnal AA-NAT mRNA levels by extension of light (800 lux white light) into the anticipated dark phase. Values represent the mean (+SEM) for the log transformed ratio of AA-NAT mRNA in mice exposed to light to that of genotype-paired, dark-exposed controls.

### DETAILED DESCRIPTION OF THE INVENTION

I. Introduction

The present invention provides non-human transgenic knockout animals that do not express a functional melanopsin protein. Such animals can exhibit attenuation of phase-shift of circadian rhythms in response to a light pulse during the dark part of an environmental dark/light cycle. The present invention also provides cells derived from the knockout animals, and methodologies for making and using these cells and knockout animals. For example, the present invention also provides methods to identify agents that specifically advance or delay the phase of circadian rhythms in humans and other animals, as well as methods of using such agents to prevent or treat conditions related to disrupted circadian rhythms. The invention also provides methods of diagnosing genetic disorders related to circadian rhythm.

II. Melanopsin Knockout Mice

The present invention provides transgenic non-human mammals that lack a functional melanopsin gene. A number of methods for making transgenic knockout animals are known in the art. Briefly, one standard methodology for producing a transgenic embryo involves introducing a targeting construct, which is designed to integrate by homologous recombination with the endogenous nucleic acid sequence of the targeted gene, into a suitable embryonic stem cells (ES). The ES cells are then cultured under conditions effective for homologous recombination (i.e., of the recombinant nucleic acid sequence of the targeting construct and the genomic nucleic acid sequence of the host cell chromosome). Genetically engineered stem cells that are identified as comprising a knockout genotype which comprises the recombinant allele is introduced into an animal at an embryonic stage using standard techniques which are well known in the art (e.g., by microinjecting the genetically engineered embryonic stem (ES) cell into a blastocyst). The resulting chimeric blastocyst is then placed within the uterus of a pseudo-pregnant foster mother for the development into viable pups. The resulting viable pups include potentially chimeric founder animals whose somatic and germline tissue comprise a mixture of cells derived from the genetically-engineered ES cells and the recipient blastocyst. The contribution of the genetically altered stem cell to the germline of the resulting chimeric mice allows the altered ES cell genome which comprises the disrupted target gene to be transmitted to the progeny of these founder animals thereby facilitating the production of transgenic “knockout animals” whose genomes comprise a gene which has been genetically engineered to comprise a particular defect in a target gene.

In a particular embodiment of the present invention, a transgenic melanopsin knockout mammal is produced by introducing a targeting vector that disrupts the melanopsin gene into an embryonic stem cell, thereby producing a transgenic stem cell. A transgenic embryonic stem cell that includes the disrupted melanopsin gene due to the integration of the targeting vector into its genome is selected and introduced into a blastocyst, thereby forming a chimeric blastocyst. The chimeric blastocyst is introduced into the uterus of a pseudopregnant mammal wherein the pseudopregnant mammal gives birth to a transgenic non-human mammal that lacks a functional melanopsin gene.

As a result of the disruption of the melanopsin gene, the melanopsin knockout mammal of the present invention can manifest a modulated ability to change its circadian rhythm. For example, in some embodiments, the melanopsin knockout mouse displays an attenuated ability to shift phase in circadian rhythm in response to a light pulse introduced in the dark part of he environmental dark/light cycle.

One embodiment of the present invention provides a vector construct (e.g., a melanopsin targeting vector or melanopsin targeting construct) designed to disrupt the function of a wild-type (endogenous) mammalian melanopsin gene. In general terms, an effective melanopsin targeting vector comprises a recombinant sequence that is effective for homologous recombination with the melanopsin gene. For example, a replacement targeting vector comprising a genomic nucleotide sequence which is homologous to the target sequence operably linked to a second nucleotide sequence which encodes a selectable marker gene exemplifies an effective targeting vector. Integration of the targeting sequence into the chromosomal DNA of the host cell (e.g., embryonic stem cell) as a result of homologous recombination introduces an intentional disruption, defect or alteration (e.g., insertion, deletion) into the sequence of the endogenous gene.

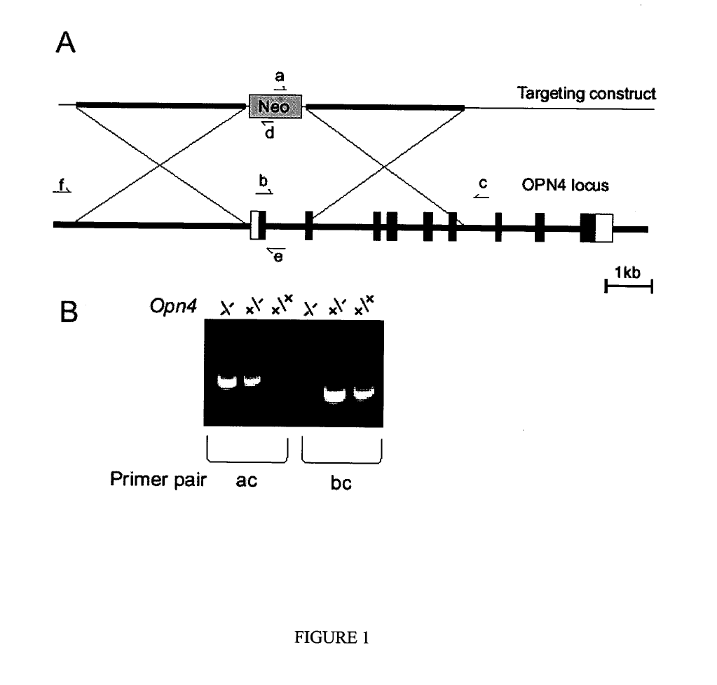

One of skill in the art will recognize that any melanopsin genomic nucleotide sequence of appropriate length and composition to facilitate homologous recombination at a specific site that has been preselected for disruption can be employed to construct a melanopsin targeting vector. Guidelines for the selection and use of sequences are described for example in Deng and Cappecchi, Mol. Cell. Biol. 12:3365-3371 (1992) and Bollag, et al., Annu. Rev. Genet. 23:199-225 (1989). For example, a wild-type melanopsin gene can be mutated and/or disrupted by inserting a recombinant nucleic acid sequence (e.g., a melanopsin targeting construct or vector) into all or a portion of the melanopsin gene locus. For example, a targeting construct can be designed to recombine with a particular portion within the enhancer, promoter, coding region, start codon, noncoding sequence, introns or exons of the melanopsin gene. In some embodiments, exon 1 of melanopsin is replaced with, e.g., a neomycin cassette. See, e.g., FIG. 1A.

One of skill in the art will readily recognize that a large number of appropriate vectors known in the art can be used as the basis of a suitable targeting vector. In practice, any vector that is capable of accommodating the recombinant nucleic acid sequence required to direct homologous recombination and to disrupt the target gene can be used. For example, pBR322, pACY164, pKK223-3, pUC8, pKG, pUC19, pLG339, pR290, pKC11 or other plasmid vectors can be used. Alternatively, a viral vector such as the lambda gt11 vector system can provide the backbone (e.g. cassette) for the targeting construct.

Basic texts disclosing general molecular biology methods of use in this invention include Sambrook et al., Molecular Cloning, A Laboratory Manual (3rd ed. 2001); Kriegler, Gene Transfer and Expression: A Laboratory Manual (1990); and Current Protocols in Molecular Biology (Ausubel et al., eds., 1994)).

According to techniques well known to those of skill in the art genetically engineered (e.g., transfected using electroporation or transformed by infection) embryonic stem cells are routinely employed for the production of transgenic non-human embryos. Embryonic stem (ES) cells are pluripotent cells isolated from the inner cell mass of mammalian blastocyst. ES cells can be cultured in vitro under appropriate culture conditions in an undifferentiated state and retain the ability to resume normal in vivo development as a result of being combined with blastocyst and introduced into the uterus of a pseudopregnant foster mother. Those of skill in the art will recognize that various stem cells are known in the art, for example AB-1, HM-1, D3. CC1.2, E-14T62a, RW4 or JI (Teratoacarcinoma and Embryonic Stem Cells: A Practical Approach, E. J. Roberston, ed., IRL Press, 1987).

The transgenic melanopsin knockout mammals described herein can also be bred (e.g., inbred, outbred or crossbred) with appropriate mates to produce colonies of animals whose genomes comprise at least one non-functional allele of the endogenous gene which naturally encodes and expresses functional melanopsin. Examples of such breeding strategies include but are not limited to: crossing of heterozygous knockout animals to produce homozygous animals; outbreeding of founder animals (e.g., heterozygous or homozygous knockouts).

III. Circadian Rhythm Modulators

Modulators of circadian rhythms are useful for preventing or treating a number of conditions by specifically advancing or delaying the phase of certain circadian rhythms in humans. The administration to a subject of an appropriate amount of a modulator of the invention is useful, for example, to achieve chronobiologic effects and/or to alleviate circadian rhythm phase disturbances in subjects in need thereof. Conditions treatable by such modulators include, e.g., insomnia, Seasonal Affective Disorder (SAD), Shift Work dysrhythmia, delayed-sleep phase syndrome (in which the major sleep episode is delayed by 2 or more hours of the desired bedtime), Irregular Sleep/Wake Pattern (characterized by irregular sleep/wake timing in which napping is prevalent and occurs irregularly throughout the daytime hours.), Advanced Sleep Phase Syndrome (characterized by intractable sleepiness during the early evening hours with awakening typically between 2 and 4 am), Non-24-hour Sleep/Wake Syndrome (characterized by intermittent insomnia that recurs with a regular periodicity over several days), and Time Zone Change Syndrome (jet lag). In addition, the modulators can be administered to, e.g., persons who live in a climate or climates which possess abnormal amounts of light or darkness; those suffering from winter depression, or other forms of depression; the aged; Alzheimer's disease patients, or those suffering from other forms of dementia; or patients who require dosages of medication at appropriate times in the circadian cycles.

In some embodiments, the subject mammal is a human. Although the present invention is applicable to both old and young people, it may find greater application in elderly people. Further, although the invention can enhance the sleep of healthy people, it can be especially beneficial for enhancing the sleep quality of people suffering from sleep disorders or sleep disturbances. In some embodiments, animals, including agriculturally important animals such as bovines or pigs, can be treated with the modulators of the invention.

Alterations of response in the knockout mouse can indicate that the agent acts on a melanopsin-specific signal transduction pathway. In some embodiments, the effect of the agent on a knockout mouse of the invention is compared to the effect of the agent on a mouse with a wild type circadian rhythm. A number of different screening protocols can be utilized to identify agents that modulate circadian rhythm in a melanopsin knockout animal of the invention. In general terms, the screening methods involve screening one or more agents to identify an agent that modulates circadian rhythm in melanopsin knockout animals.

For example, the agents can be tested for their ability to affect phase-shift of melanopsin knockout animals in response to light. Typically a pulse of light is applied in a dark period of an animal's cycle. The length of the pulse can vary. In some embodiments, the pulse of light has a duration between 10 minutes and 2 hours. The timing of the pulse will also cause a different effect on the phase-shift in the circadian rhythm. For example, a pulse in the early part of a dark period can lead to the opposite effect (i.e., a shift earlier) of a pulse provided late in the dark period (e.g., resulting in a shift later).

Finally, the quality of the light pulse can vary. In some embodiments, the intensity of the light pulse is low. Low intensity light is typically between 0.001 and 1.5 lux. In other embodiments, a high intensity light is used, e.g., greater than 1.5 lux. In some embodiments, the light is monochromatic, typically around 480 nm.

In some embodiments, the pulse of light is introduced into the point of an animal's cycle that is typically dark (e.g., night) and the effect of the pulse is monitored with respect to a marker of circadian rhythm. An agent is administered to the animal, and the effect of the agent on circadian rhythm and phase-shift is then monitored. Common examples of circadian rhythm markers include locomotor (e.g., animal activity), core body temperature or melatonin levels. Alternatively, the effect of an agent on melatonin production in a wildtype or melanopsin knockout mouse can be determined. Exemplary melatonin responses to light are described in, e.g., Illnerova et al., Comp. Biochem. Physiol. A74:155-159 (1983); Vakkuri et al., J. Endocrinol. 105:263-268 (1985); and Maitra et al., Eur. Arch. Biol. 103:157-164 (1992).

The agents tested as circadian rhythm modulators of the invention can be any small chemical compound, or a biological entity, such as a protein, sugar, nucleic acid or lipid. In some embodiments, nucleic acid libraries (e.g., cDNA libraries) are expressed in transgenic melanopsin knockout animals or their cells. Alternatively, test compounds will be small organic molecules or peptides. Essentially any chemical compound can be used as a potential modulator or ligand in the assays of the invention, although most often compounds that can be dissolved in aqueous or organic (especially DMSO-based) solutions are used. The assays are designed to screen large chemical libraries by automating the assay steps and providing compounds from any convenient source to assays, which are typically run in parallel (e.g., in microtiter formats on microtiter plates in robotic assays). Modulators can also include agents designed to reduce the level of mRNA (e.g. antisense molecules, ribozymes, DNAzymes, small inhibitory RNAs and the like). It will be appreciated that there are many suppliers of chemical compounds, including Sigma (St. Louis, Mo.), Aldrich (St. Louis, Mo.), Sigma-Aldrich (St. Louis, Mo.), Fluka Chemika-Biochemica Analytika (Buchs, Switzerland) and the like.

In some embodiments, high throughput screening methods involve providing a combinatorial chemical or peptide library containing a large number of potential therapeutic compounds (potential modulator compounds). Such “combinatorial chemical libraries” or “ligand libraries” are then screened in one or more assays, as described herein, to identify those library members (particular chemical species or subclasses) that display a desired characteristic activity. The compounds thus identified can serve as conventional “lead compounds” or can themselves be used as potential or actual therapeutics.

A combinatorial chemical library is a collection of diverse chemical compounds generated by either chemical synthesis or biological synthesis, by combining a number of chemical “building blocks” such as reagents. For example, a linear combinatorial chemical library such as a polypeptide library is formed by combining a set of chemical building blocks (amino acids) in every possible way for a given compound length (i.e., the number of amino acids in a polypeptide compound). Millions of chemical compounds can be synthesized through such combinatorial mixing of chemical building blocks.

Preparation and screening of combinatorial chemical libraries is well known to those of skill in the art. Such combinatorial chemical libraries include, but are not limited to, peptide libraries (see, e.g., U.S. Pat. No. 5,010,175, Furka, Int. J. Pept. Prot. Res. 37:487-493 (1991) and Houghton et al., Nature 354:84-88 (1991)). Other chemistries for generating chemical diversity libraries can also be used. Such chemistries include, but are not limited to: peptoids (e.g., PCT Publication No. WO 91/19735), encoded peptides (e.g., PCT Publication WO 93/20242), random bio-oligomers (e.g., PCT Publication No. WO 92/00091), benzodiazepines (e.g., U.S. Pat. No. 5,288,514), diversomers such as hydantoins, benzodiazepines and dipeptides (Hobbs et al., Proc. Nat. Acad. Sci. USA 90:6909-6913 (1993)), vinylogous polypeptides (Hagihara et al., J. Amer. Chem. Soc. 114:6568 (1992)), nonpeptidal peptidomimetics with glucose scaffolding (Hirschmann et al., J. Amer. Chem. Soc. 114:9217-9218 (1992)), analogous organic syntheses of small compound libraries (Chen et al., J. Amer. Chem. Soc. 116:2661 (1994)), oligocarbamates (Cho et al., Science 261:1303 (1993)), and/or peptidyl phosphonates (Campbell et al., J. Org. Chem. 59:658 (1994)), nucleic acid libraries (see Ausubel, Berger and Sambrook, all supra), peptide nucleic acid libraries (see, e.g., U.S. Pat. No. 5,539,083), antibody libraries (see, e.g., Vaughn et al., Nature Biotechnology, 14(3):309-314 (1996) and PCT/US96/10287), carbohydrate libraries (see, e.g., Liang et al., Science, 274:1520-1522 (1996) and U.S. Pat. No. 5,593,853), small organic molecule libraries (see, e.g., benzodiazepines, Baum C&EN, January 18, page 33 (1993); isoprenoids, U.S. Pat. No. 5,569,588; thiazolidinones and metathiazanones, U.S. Pat. No. 5,549,974; pyrrolidines, U.S. Pat. Nos. 5,525,735 and 5,519,134; morpholino compounds, U.S. Pat. No. 5,506,337; benzodiazepines, U.S. Pat. No. 5,288,514, and the like).

Devices for the preparation of combinatorial libraries are commercially available (see, e.g., 357 MPS, 390 MPS, Advanced Chem Tech, Louisville Ky., Symphony, Rainin, Woburn, Mass., 433A Applied Biosystems, Foster City, Calif., 9050 Plus, Millipore, Bedford, Mass.). In addition, numerous combinatorial libraries are themselves commercially available (see, e.g., ComGenex, Princeton, N.J., Tripos, Inc., St. Louis, Mo., 3D Pharmaceuticals, Exton, Pa., Martek Biosciences, Columbia, Md., etc.).

Preliminary screens to identify potential modulators of circadian rhythm can be conducted by screening for agents capable of binding to melanopsin or other polypeptides that play a role in circadian rhythm. Binding assays usually involve contacting melanopsin or another polypeptide implicated in circadian rhythm with one or more test agents and allowing sufficient time for the protein and test agents to form a binding complex. Any binding complexes formed can be detected using any of a number of established analytical techniques. Protein binding assays include, but are not limited to, methods that measure co-precipitation or co-migration on non-denaturing SDS-polyacrylamide gels, and co-migration on Western blots (see, e.g., Bennet, J. P. and Yamamura, H. I. (1985) “Neurotransmitter, Hormone or Drug Receptor Binding Methods,” in Neurotransmitter Receptor Binding (Yamamura, H. I., et al., eds.), pp. 61-89. Other binding assays involve the use of mass spectrometry or NMR techniques to identify molecules bound to a polypeptide of the invention or displacement of labeled substrates. The polypeptides of the invention utilized in such assays can be naturally expressed, cloned or synthesized.

In addition, mammalian or yeast two-hybrid approaches (see, e.g., Bartel, P. L. et. al. Methods Enzymol, 254:241 (1995)) can be used to identify polypeptides or other molecules that interact or bind when expressed together in a host cell.

Samples or assays that are treated with a potential modulator (e.g., a “test compound”) are compared to control samples without the test compound, to examine the extent of modulation. Control samples (untreated with activators or inhibitors) are assigned a relative activity value of 100. Inhibition of a polypeptide is achieved when the activity value relative to the control is about 90%, optionally 50%, optionally 25-0%. Activation of a polypeptide is achieved when the activity value relative to the control is 110%, optionally 150%, 200%, 300%, 400%, 500%, or 1000-2000%.

IV. Administration and Pharmaceutical Compositions

Circadian rhythm modulators of the invention can be administered directly to the mammalian subject. Administration is by any of the routes normally used for introducing a modulator compound into ultimate contact with the tissue to be treated and is well known to those of skill in the art.

The pharmaceutical compositions of the invention may comprise a pharmaceutically acceptable carrier. Pharmaceutically acceptable carriers are determined in part by the particular composition being administered, as well as by the particular method used to administer the composition. Accordingly, there are a wide variety of suitable formulations of pharmaceutical compositions of the present invention (see, e.g., Remington's Pharmaceutical Sciences, 17^{th} ed. 1985)).

Formulations suitable for administration include aqueous and non-aqueous solutions, isotonic sterile solutions, which can contain antioxidants, buffers, bacteriostats, and solutes that render the formulation isotonic, and aqueous and non-aqueous sterile suspensions that can include suspending agents, solubilizers, thickening agents, stabilizers, and preservatives. In the practice of this invention, compositions can be administered, for example, orally, nasally, topically, intravenously, intraperitoneally, intrathecally or into the eye (e.g., by eye drop or injection). The formulations of compounds can be presented in unit-dose or multi-dose sealed containers, such as ampoules and vials. Solutions and suspensions can be prepared from sterile powders, granules, and tablets of the kind previously described. The modulators can also be administered as part of a prepared food or drug.

The dose administered to a patient, in the context of the present invention should be sufficient to induce a beneficial response in the subject over time, i.e., to modulate the circadian rhythm of the subject. The optimal dose level for any patient will depend on a variety of factors including the efficacy of the specific modulator employed, the age, body weight, physical activity, and diet of the patient, and on a possible combination with other drug. The size of the dose also will be determined by the existence, nature, and extent of any adverse side-effects that accompany the administration of a particular compound or vector in a particular subject.

In determining the effective amount of the modulator to be administered a physician may evaluate circulating plasma levels of the modulator, modulator toxicity, and the production of anti-modulator antibodies. In general, the dose equivalent of a modulator is from about 1 ng/kg to 10 mg/kg for a typical subject.

For administration, modulators of the present invention can be administered at a rate determined by the LD-50 of the modulator, and the side-effects of the modulator at various concentrations, as applied to the mass and overall health of the subject. Administration can be accomplished via single or divided doses.

The modulators of the invention may be used alone or in conjunction with other agents that are known to be beneficial in altering circadian rhythms or in the enhancement of sleep efficiency. The circadian modulators of the invention and an other agent may be coadministered, either in concomitant therapy or in a fixed combination, or they may be administered at separate times. For example, the circadian modulators of the invention may be administered in conjunction with other compounds which are known in the art to be useful for suppressing or stimulating melatonin production including melatonergic agents, noradrenergic and serotonergic re-uptake blockers, alpha-1-noradrenergic agonists, monamine oxidase inhibitors, neuropeptide Y agonists or antagonists; neurokinin-1 agonists; substance P; beta-adrenergic blockers and benzodiazepines, such as atenolol; or with other compounds which are known in the art to be useful for stimulating melatonin production including tricyclic antidepressants and alpha-2-adrenergic antagonists; or with melatonin precursors such as tryptophan, 5-hydroxytryptophan, serotonin and N-acetylserotonin; as well as melatonin analogs, melatonin agonists and melatonin antagonists, or melatonin itself. In addition, the circadian modulators of the invention may be administered in conjunction with other compounds which are known in the art to be useful for enhancing sleep quality and preventing and treating sleep disorders and sleep disturbances, including e.g., sedatives, hypnotics, anxiolytics, antipsychotics, antianxiety agents, minor tranquilizers, melatonin agonists and antagonists, melatonin, melatonergic agents, benzodiazepines, barbituates, 5HT-2 antagonists, and the like, such as: adinazolam, allobarbital, alonimid, alprazolam, amitriptyline, amobarbital, amoxapine, bentazepam, benzoctamine, brotizolam, bupropion, busprione, butabarbital, butalbital, capuride, carbocloral, chloral betaine, chloral hydrate, chlordiazepoxide, clomipramine, cloperidone, clorazepate, clorethate, clozapine, cyprazepam, desipramine, dexclamol, diazepam, dichloralphenazone, divalproex, diphenhydramine, doxepin, estazolam, ethchlorvynol, etomidate, fenobam, flunitrazepam, flurazepam, fluvoxamine, fluoxetine, fosazepam, glutethimide, halazepam, hydroxyzine, imipramine, lithium, lorazepam, lormetazepam, maprotiline, mecloqualone, melatonin, mephobarbital, meprobamate, methaqualone, midaflur, midazolam, nefazodone, nisobamate, nitrazepam, nortriptyline, oxazepam, paraldehyde, paroxetine, pentobarbital, perlapine, perphenazine, phenelzine, phenobarbital, prazepam, promethazine, propofol, protriptyline, quazepam, reclazepam, roletamide, secobarbital, sertraline, suproclone, temazepam, thioridazine, tracazolate, tranylcypromaine, trazodone, triazolam, trepipam, tricetamide, triclofos, trifluoperazine, trimetozine, trimipramine, uldazepam, valproate, venlafaxine, zaleplon, zolazepam, zolpidem, and salts thereof, and combinations thereof, and the like.

The circadian modulators of the invention may be administered in conjunction with the use of physical methods such as with light therapy or electrical stimulation. In particular, the circadian modulators of the invention may be administered in conjunction with scheduling bright light administration, ordinary-intensity light exposure, or exposure to dim-light or darkness (or even sleep).

V. Diagnosis of Genetic Circadian Rhythm Disorders

The invention provides methods of diagnosing familial disorders associated with melanopsin. Genetic disorders associated with melanopsin can include, e.g., familial circadian rhythm disorders, including, e.g., familial delayed-sleep phase disorder. Diagnosis will typically involve detection of an individual's melanopsin gene or gene products (RNA, protein) for an alteration of structure or expression compared to a normal control individual's structure or expression.

A variety of methods of specific DNA and RNA measurement that use nucleic acid hybridization techniques are known to those of skill in the art (see, Sambrook, supra). Some methods involve an electrophoretic separation (e.g., Southern blot for detecting DNA, and Northern blot for detecting RNA), but measurement of DNA and RNA can also be carried out in the absence of electrophoretic separation (e.g., by dot blot). Southern blot of genomic DNA (e.g., from a human) can be used for screening for restriction fragment length polymorphism (RFLP) to detect the presence of a genetic disorder affecting a polypeptide of the invention.

A variety of nucleic acid hybridization formats are known to those skilled in the art. For example, common formats include sandwich assays and competition or displacement assays. Hybridization techniques are generally described in Hames and Higgins Nucleic Acid Hybridization, A Practical Approach, IRL Press (1985); Gall and Pardue, Proc. Natl. Acad. Sci. U.S.A., 63:378-383 (1969); and John et al. Nature, 223:582-587 (1969).

Detection of a hybridization complex may require the binding of a signal-generating complex to a duplex of target and probe polynucleotides or nucleic acids. Typically, such binding occurs through ligand and anti-ligand interactions as between a ligand-conjugated probe and an anti-ligand conjugated with a signal. The binding of the signal generation complex is also readily amenable to accelerations by exposure to ultrasonic energy.

The label may also allow indirect detection of the hybridization complex. For example, where the label is a hapten or antigen, the sample can be detected by using antibodies. In these systems, a signal is generated by attaching fluorescent or enzyme molecules to the antibodies or in some cases, by attachment to a radioactive label (see, e.g., Tijssen, “Practice and Theory of Enzyme Immunoassays,” Laboratory Techniques in Biochemistry and Molecular Biology, Burdon and van Knippenberg Eds., Elsevier (1985), pp. 9-20).

The probes are typically labeled either directly, as with isotopes, chromophores, lumiphores, chromogens, or indirectly, such as with biotin, to which a streptavidin complex may later bind. Thus, the detectable labels used in the assays of the present invention can be primary labels (where the label comprises an element that is detected directly or that produces a directly detectable element) or secondary labels (where the detected label binds to a primary label, e.g., as is common in immunological labeling). Typically, labeled signal nucleic acids are used to detect hybridization. Complementary nucleic acids or signal nucleic acids may be labeled by any one of several methods typically used to detect the presence of hybridized polynucleotides. The most common method of detection is the use of autoradiography with ³H, ¹²⁵I, ³⁵S, ¹⁴C, or ³²P-labeled probes or the like.

Other labels include, e.g., ligands that bind to labeled antibodies, fluorophores, chemiluminescent agents, enzymes, and antibodies that can serve as specific binding pair members for a labeled ligand. An introduction to labels, labeling procedures and detection of labels is found in Polak and Van Noorden Introduction to Immunocytochemistry, 2nd ed., Springer Verlag, NY (1997); and in Haugland Handbook of Fluorescent Probes and Research Chemicals, a combined handbook and catalogue Published by Molecular Probes, Inc. (1996).

In general, a detector that monitors a particular probe or probe combination is used to detect the detection reagent label. Typical detectors include spectrophotometers, phototubes and photodiodes, microscopes, scintillation counters, cameras, film and the like, as well as combinations thereof. Examples of suitable detectors are widely available from a variety of commercial sources known to persons of skill in the art. Commonly, an optical image of a substrate comprising bound labeling moieties is digitized for subsequent computer analysis.

The amount of, for example, an RNA is measured by quantifying the amount of label fixed to the solid support by binding of the detection reagent. Typically, the presence of a modulator during incubation will increase or decrease the amount of label fixed to the solid support relative to a control incubation that does not comprise the modulator, or as compared to a baseline established for a particular reaction type. Means of detecting and quantifying labels are well known to those of skill in the art.

In some embodiments, the target nucleic acid or the probe is immobilized on a solid support. Solid supports suitable for use in the assays of the invention are known to those of skill in the art. As used herein, a solid support is a matrix of material in a substantially fixed arrangement.

A variety of automated solid-phase assay techniques are also appropriate. For instance, very large scale immobilized polymer arrays (VLSIPS™), i.e. Gene Chips or microarrays, available from Affymetrix, Inc. in Santa Clara, Calif. can be used to detect changes in expression levels of a plurality of genes involved in the same regulatory pathways simultaneously. See, Tijssen, supra., Fodor et al. (1991) Science, 251: 767-777; Sheldon et al. (1993) Clinical Chemistry 39(4): 718-719, and Kozal et al. (1996) Nature Medicine 2(7): 753-759. Similarly, spotted cDNA arrays (arrays of cDNA sequences bound to nylon, glass or another solid support) can also be used to monitor expression of a plurality of genes.

Detection of nucleic acids can also be accomplished, for example, by using a labeled detection moiety that binds specifically to duplex nucleic acids (e.g., an antibody that is specific for RNA-DNA duplexes). One example uses an antibody that recognizes DNA-RNA heteroduplexes in which the antibody is linked to an enzyme (typically by recombinant or covalent chemical bonding). The antibody is detected when the enzyme reacts with its substrate, producing a detectable product. Coutlee et al. (1989) Analytical Biochemistry 181:153-162; Bogulavski (1986) et al. J. Immunol. Methods 89:123-130; Prooijen-Knegt (1982) Exp. Cell Res. 141:397-407; Rudkin (1976) Nature 265:472-473, Stollar (1970) PNAS 65:993-1000; Ballard (1982) Mol. Immunol. 19:793-799; Pisetsky and Caster (1982) Mol. Immunol. 19:645-650; Viscidi et al. (1988) J. Clin. Microbial. 41:199-209; and Kiney et al. (1989) J. Clin. Microbiol. 27:6-12 describe antibodies to RNA duplexes, including homo and heteroduplexes. Kits comprising antibodies specific for DNA:RNA hybrids are available, e.g., from Digene Diagnostics, Inc. (Beltsville, Md.).

In addition to available antibodies, one of skill in the art can easily make antibodies specific for nucleic acid duplexes using existing techniques, or modify those antibodies that are commercially or publicly available. In addition to the art referenced above, general methods for producing polyclonal and monoclonal antibodies are known to those of skill in the art (see, e.g., Paul (ed) Fundamental Immunology, Third Edition Raven Press, Ltd., NY (1993); Coligan Current Protocols in Immunology Wiley/Greene, NY (1991); Harlow and Lane Antibodies: A Laboratory Manual Cold Spring Harbor Press, NY (1989); Stites et al. (eds.) Basic and Clinical Immunology (4th ed.) Lange Medical Publications, Los Altos, Calif., and references cited therein; Goding Monoclonal Antibodies: Principles and Practice (2d ed.) Academic Press, New York, N.Y., (1986); and Kohler and Milstein Nature 256: 495-497 (1975)). Other suitable techniques for antibody preparation include selection of libraries of recombinant antibodies in phage or similar vectors (see, Huse et al. Science 246:1275-1281 (1989); and Ward et al. Nature 341:544-546 (1989)). Specific monoclonal and polyclonal antibodies and antisera will usually bind with a K_{D} of at least about 0.1 μM, preferably at least about 0.01 μM or better, and most typically and preferably, 0.001 μM or better.

The nucleic acids used in this invention can be either positive or negative probes. Positive probes bind to their targets and the presence of duplex formation is evidence of the presence of the target. Negative probes fail to bind to the suspect target and the absence of duplex formation is evidence of the presence of the target. For example, the use of a wild type specific nucleic acid probe or PCR primers may serve as a negative probe in an assay sample where only the nucleotide sequence of interest is present.

The sensitivity of the hybridization assays may be enhanced through use of a nucleic acid amplification system that multiplies the target nucleic acid being detected. Examples of such systems include the polymerase chain reaction (PCR) system and the ligase chain reaction (LCR) system. Other methods recently described in the art are the nucleic acid sequence based amplification (NASBA, Cangene, Mississauga, Ontario) and Q Beta Replicase systems. These systems can be used to directly identify mutants where the PCR or LCR primers are designed to be extended or ligated only when a selected sequence is present. Alternatively, the selected sequences can be generally amplified using, for example, nonspecific PCR primers and the amplified target region later probed for a specific sequence indicative of a mutation. It is understood that various detection probes, including Taqman and molecular beacon probes can be used to monitor amplification reaction products, e.g., in real time.

An alternative means for determining the level of expression of the nucleic acids of the present invention is in situ hybridization. In situ hybridization assays are well known and are generally described in Angerer et al., Methods Enzymol. 152:649-660 (1987). In an in situ hybridization assay, cells, preferentially human cells from the cerebellum or the hippocampus, are fixed to a solid support, typically a glass slide. If DNA is to be probed, the cells are denatured with heat or alkali. The cells are then contacted with a hybridization solution at a moderate temperature to permit annealing of specific probes that are labeled. The probes are preferably labeled with radioisotopes or fluorescent reporters.

Single nucleotide polymorphism (SNP) analysis is also useful for detecting differences between alleles of the polynucleotides (e.g., genes) of the invention. SNPs linked to genes encoding polypeptides of the invention are useful, for instance, for diagnosis of diseases whose occurrence is linked to the gene sequences of the invention. For example, if an individual carries at least one SNP linked to a disease-associated allele of the gene sequences of the invention, the individual is likely predisposed for one or more of those diseases. If the individual is homozygous for a disease-linked SNP, the individual is particularly predisposed for occurrence of that disease.

All publications and patent applications cited in this specification are herein incorporated by reference as if each individual publication or patent application were specifically and individually indicated to be incorporated by reference.

Although the foregoing invention has been described in some detail by way of illustration and example for purposes of clarity of understanding, it will be readily apparent to one of ordinary skill in the art in light of the teachings of this invention that certain changes and modifications may be made thereto without departing from the spirit or scope of the appended claims.

### EXAMPLES

The following examples are offered to illustrate, but not to limit the claimed invention.

To formally investigate melanopsin's role in light resetting of the circadian clock in mammals, we have generated melanopsin-null mice. Characterization of these mice revealed that the animals display normal circadian locomotor activity rhythms, but are deficient in their phase shifting response to light.

To generate melanopsin knockout mice, we replaced exon 1 of melanopsin with a neomycin gene by homologous recombination in embryonic stem cells (FIG. 1A). A targeting construct was generated by cloning a 3.1 kb 5′ arm and a 3.2 kb 3'arm of genomic DNA (FIG. 1A) from a BAC clone encompassing the Opn4 locus into a modified version of pGEM3 (Promega, Madison, Wis.). The targeting construct was linearized by NotI, and microinjected into an embryonic stem (ES) cell line from 129S1/Sv. The ES cell clones were selected on G418, and 96 positive ES cell clones were screened by PCR using primer pairs ac and df for replacement of the exonl with neomycin resistance gene. Two clones with appropriate insertion were injected into C57/B16 blastocysts and introduced into C57/B16 pseudopregnant females. Chimeric males were mated with C57/B16 females. Chimeras from a single clone produced agouti coat color heterozygote animals, which were subsequently mated with C57/B16 mice. Heterozygote mice were interbred, and the resultant progeny were genotyped by PCR amplification. The progeny were found to have a normal 1:2:1 Mendelian segregation pattern for the Opn4^{neo} allele, suggesting that Opn4 is not required for normal viability. Opn4^{-/-} mice and littermate Opn4^{+/-}, and Opn4^{+/+} mice were used in all assays. Primer a=CAGGAGCAAGGTGAGATGACAGGAG, b=AGGATGGTATAGAGCCGGAAGTCTG, c=TCAAGCCACAGAGGATACTAGCAGG, d=GATGATCTGGACGAAGAGCATCAGG, e=ACTGAGGACTGACACTGAAGCCTGG, f=CAGTGTCAGGCCTAGCGGGAAGAGA.

The targeted locus exhibits normal autosomal Mendelian inheritance, and the homozygous knockout mice (Opn4^{-/-}) are apparently healthy with normal eyes and no visible developmental defects. The general architecture of the retina and gross cellular morphology of different constituent cells also showed no detectable defect. Melanopsin-positive retinal ganglion cells are present in flat-mounts of melanopsin^{+/+} and melanopsin^{+/+} mouse retinas, but not in those of melanopsin^{-/-} mice. Immunocytochemistry of the retina revealed no immunostaining for melanopsin in Opn4^{-/-} mice, validating both the targeting strategy and the antibodies (I. Provencio, M. D. Rollag, A. M. Castrucci, Nature 415:493. (2002)) employed. In contrast, littermate Opn4^{+/+} and Opn4^{+/-} mice had anti-melanopsin immunoreactive RGCs (FIG. 1C-H) identical to those previously described in mice, and rats (Hannibal, et al., J Neurosci 22:RC191. (2002); S. Hattar, et al., Science 295:1065-70. (2002). These cells possessed heavily labeled axons and broad immunoreactive dendritic arbors that terminated in the outer sublamina of the inner plexiform layer and heavily immunoreactive axons. Immunoreactivity can be eliminated by pre-adsorbing the antiserum with 100 ng/ml of the immunizing peptide (see supplementary information).

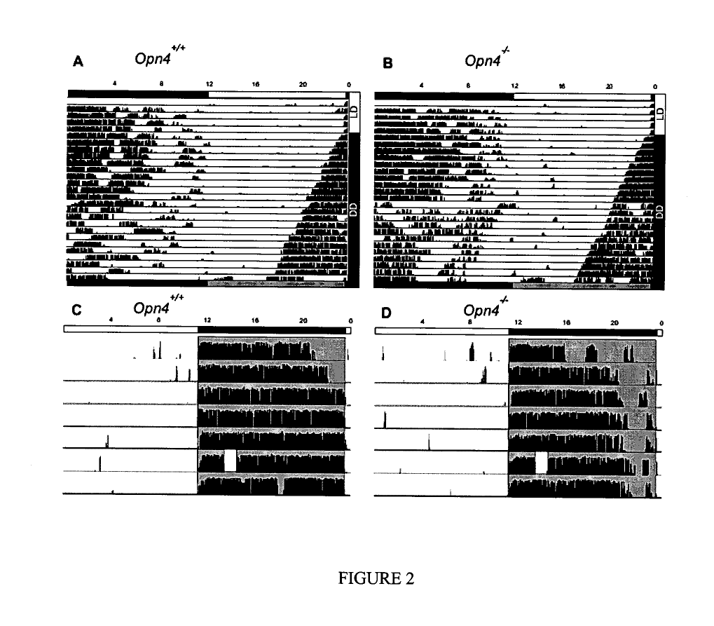

We next sought to characterize locomotor activity rhythms in driven and free-running conditions in the Opn4^{-/-} mice. This analysis revealed that these mice entrained normally to the 12 h light: 12 h dark (LD) cycle, and exhibited no detectable defect in locomotor activity rhythms when placed in constant darkness. During entrainment, the phase angle of activity onset in relation to the LD cycle was similar in both the wild type and the knockout animals. In constant darkness (DD), the free running period length of the locomotor activity rhythm in the knockout mice was not significantly different from that of the wild type or heterozygous littermates (FIGS. 2A and 2B). The total activity and the length of the activity phase during a circadian cycle is also similar in all three genotypes, suggesting no significant defect in the functioning of the core oscillator, nor in the output of the clock regulating locomotor activity rhythms. Taken together, these data suggest that melanopsin does not participate in the normal functioning of the core oscillator. To test whether light masking of activity is contributing to the photoentrainment of the Opn4^{-/-} mice, we entrained the animals to a normal LD cycle and subjected them to 1 hour of 300 lux white light during the dark phase. This analysis revealed normal activity suppression by light in both the wild type and melanopsin null mice (FIG. 2C, and 2D).

To test the role of melanopsin in direct light input to the clock, we evaluated the phase shifting effect of a brief pulse of light on activity rhythms in constant darkness. The phase of activity rhythms of mice in DD changes in response to brief illumination in a well-defined, circadian phase-dependent manner. A light pulse during the subjective day produces small phase shifts, while the same illumination in early subjective night leads to large delays, and a light pulse in the late subjective night causes significant phase advances, constituting a phase response curve (PRC) (P. S. Pittendrigh, in Handbook of Behavioral Neurobiology J. Aschoff, Ed. (Plenum Press, New York, 1981), vol. 4, pp. 95-124). Previous studies have demonstrated that melanopsin positive RGCs have an action spectrum with a peak at 480 nm (S. Hattar, et al., Science 295:1065-70. (2002); D. M. Berson, et al., Science 295:1070-3. (2002)), coincident with the behavioral action spectrum in rd mice (T. Yoshimura, S. Ebihara, J Comp Physiol 178:797-802. (1996)). Therefore, we hypothesized that any light input defect in the melanopsin knockout mice would be most pronounced at this wavelength of light administered during early subjective night. We administered a 15 minute pulse of monochromatic light (480 nm with a 10 nm half-peak bandwidth) of varying irradiance 3 hours after the activity onset under constant darkness (CT15). We then evaluated the phase shifting effect of the light pulse on the phase of activity onset over subsequent days.

Under constant temperature of 21° C., 7-10 weeks old mice in wheel running cages were entrained to 12 hours white light (800 lux), 12 hours of darkness (LD) for 7-10 days and then allowed to free run in constant dark. After 7-12 days of free run, mice were given a 15 minute pulse of monochromatic light at one of three irradiances at CT15 in a light pulse apparatus, and then returned to the wheel cages. Activity was recorded, and the activity records were analyzed by the Clocklab software package. Light pulse apparatus was custom built by Enlightened Technologies Associates, Inc., Fairfax, Va. Light from a 150 W xenon lamp was collimated, filtered through narrow bandpass filters (half peak width of 10 nm) and neutral density filters, and transmitted through fiber-optic cables. At the top of a light tight chamber, the fiber-optic cables branch outwards and deliver the light in an array of termini, which then passes through a diffuser, illuminating a 15 cm tall, 10 cm diameter cylindrical chamber coated with a highly reflective white paint. Interference and neutral density filters from (Oriel Corp., Stratford, Conn.) were used to control spectral quality and fluence rate. A model 211 detector attached to a S371 optical power meter from UDT instruments, Maryland, USA was used to measure light intensity at specified wavelengths.

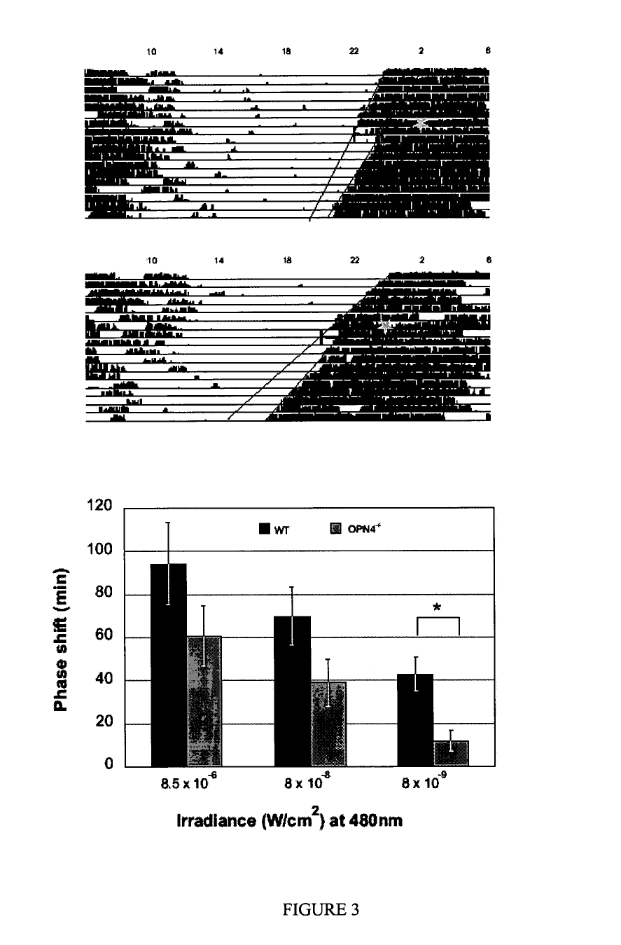

The melanopsin knockout mice exhibited a significantly attenuated phase delay in comparison to the wild type animals (FIG. 3). The phase delay was significant at subsaturating irradiance of light, while at higher irradiance, we observed only a slight attenuation of the phase shift in the knockout animals. Because the Opn4^{-/-} mice display a normal locomotor activity period length in constant darkness, the difference in the phase shifting response is unlikely to be due to a difference in the phase of the oscillator when the light pulse was administered. Therefore, we conclude that the attenuation in phase shifting is a direct result of reduced sensitivity of the photic input pathway to light in the melanopsin knockout mice.

The importance of melanopsin as a circadian photoreceptor is best appreciated in the context of nature. Nocturnal mammals such as the mouse spend most of their day in burrows in near complete darkness. It is thought that dim twilight is most likely natural signal to exert effects on light resetting of the clock in these animals. Conversely, in diurnal mammals, low irradiance light in the dawn initiates daily phase adjustments. The significant contribution of melanopsin to entrainment under low irradiance highlights its importance as a circadian photoreceptor. Equally important is the observation that other photoperceptive systems also play an important role under different lighting conditions, underscoring the complexity of circadian photoperception in mammals.

The eye is the principal mediator of light input to the central nervous system in mammals. Besides vision, the eye also mediates several non-visual responses to light, including photoentrainment of the circadian oscillator, constriction of the pupil, acute suppression of pineal melatonin, acute suppression of activity (masking) in nocturnal mammals, and regulation of sleep latency. Many of these responses persist in mice that are visually blind from outer retinal degeneration, but are abolished by bilateral enucleation of the eyes (reviewed in Foster and Hankins, Prog Retin Eye Res 21:507-27, 2002). These findings demonstrate the presence of inner retinal, non-visual ocular photoreceptor(s) specifically subserving these non-visual photic responses.

Intrinsically photosensitive retinal ganglion cells (ipRGCs) (Hattar, et al., Science 295:1065-70, 2002; and Berson et al., Science 295:1070-3, 2002) project to brain sites that mediate many of these ocular, yet non-visual responses to light including the suprachiasmatic nucleus (SCN), the intergeniculate leaflet (IGL), and the olivary pretectal nucleus that mediate pupillary light reflexes (PLR) (Foster and Hankins, Prog Retin Eye Res 21:507-27, 2002). The photosensitivity of these cells ex vivo depends on the presence of melanopsin (Lucas, et al., Science 299, 245-7, 2003).

While Opn4^{-/-} exhibit attenuation in light-induced phase resetting of the circadian oscillator, and a reduced papillary light reflex (PLR) under high irradiance levels, most non-visual photic responses in these mice remain largely intact. This suggests either the presence of additional inner retinal photoreceptors, or contributions from the outer retinal classical photoreceptors to non-visual photoresponses. To test the latter hypothesis, we generated mice deficient in both melanopsin and classical photoreceptors by breeding Opn4^{-/-} mice with the C3H/HeJ mouse strain that carries the retinal degeneration (rd) mutation.

Opn4^{-/-} mice of 129S1/SvImJ background were bred to C3H/HeJ strain carrying Pde6b^{rdl} mutation (The Jackson Laboratory, Bar Harbor). The resulting F1 progeny heterozygous at Opn4, and Pde6b loci were interbred to produce F2 progeny. The F2 progeny were genotyped at Pde6b, and a marker closely linked to Opn4 locus by the MassEXTEND genotyping method (Wiltshire, et al., Proc Natl Acad Sci USA 100:3380-5, 2003). Appropriate genotypes at Opn4 and Pde6b loci were reconfirmed by PCR.

Mice homozygous for the rd allele are visually blind due to a primary degeneration of the rods and a secondary loss of cones, but they retain melanopsin-containing RGCs. The Opn4^{-/-}; rd/rd mice were healthy and viable with intact optic nerves. Outer retinal degeneration was indistinguishable between rd/rd and Opn4^{-/-}; rd/rd mice (data not shown).

Behavioural analysis was performed as follows. Mice of 6-10 weeks in age were tested for light entrainment of circadian wheel running activity. Light intensity inside the wheel running chamber was adjusted to the desired level by wrapping the fluorescent white light source with neutral density acetate filters. Mice were entrained to 8 hr of light (100 lux or 800 lux) and 16 hrs of darkness (LD::8:16), or 12 hr of light (800 lux) and 12 hr of darkness for 2-3 weeks, and then maintained in constant darkness or in constant light (100 lux) for an additional 2-3 weeks. Acute light suppression of wheel running activity was tested on animals held at LD::8:16 with 800 lux light during the light phase. Typically 2-3 hr after lights were off, a 2 hr pulse of white light of 800 or 100 lux was administered. After at least 3 days the same mice received another light pulse of a different intensity. Since the Opn4−/−; rd/rd mice do not photoentrain, data from mice receiving the light pulse within the first 5 hours of activity onset were used for comparison. Pupillary light reflex was assessed as described in (Van Gelder, et al., Science 299:222, 2003). Irradiance-response curves were fit with a 4-parameter sigmoidal model (SigmaPlot 2000, SPSS Science, Chicago, Ill.). N=5-10 for all data points.

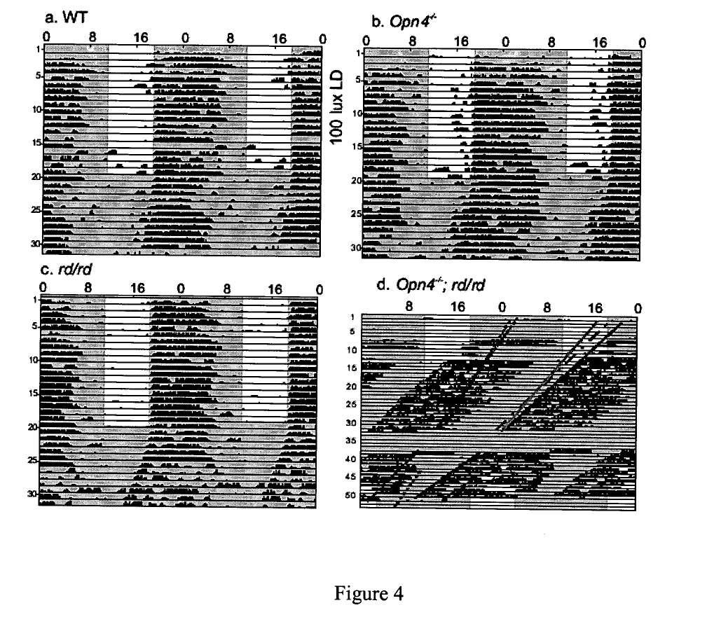

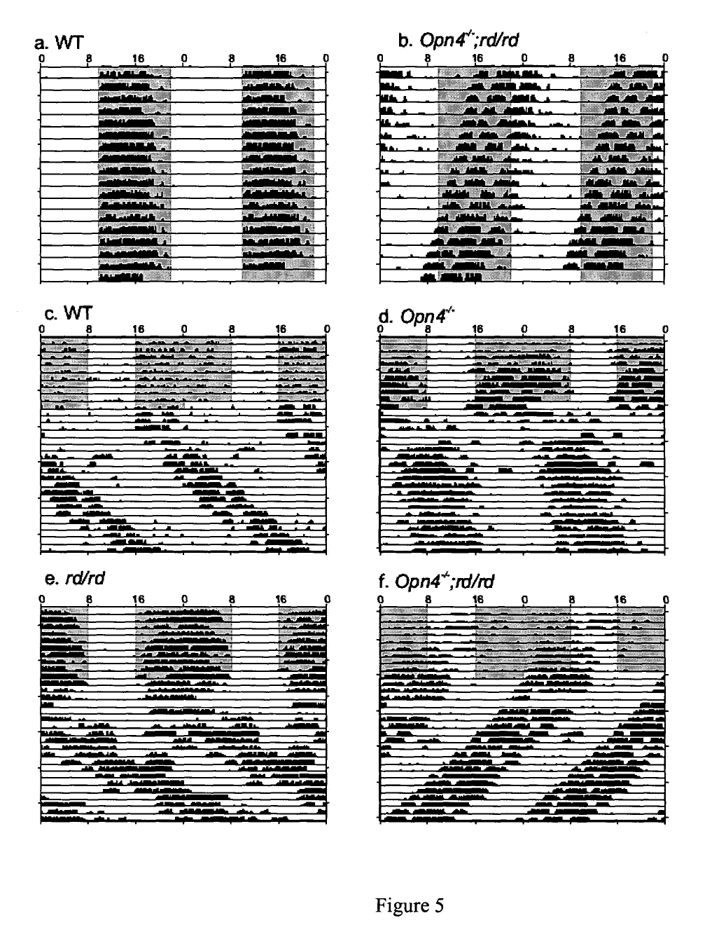

To assess the circadian photoentrainment and acute light suppression of activity, we subjected the Opn4^{-/-}; rd/rd mice, littermate wildtype (WT), rd/rd, and Opn4^{-/-} mice to a 24 hour light:dark (LD) cycle (8L: 16D). Under conditions of constant darkness (DD), mice have a free-running circadian locomotor period of less than 24 hours. However, in a 24-hour LD cycle, photic input to the oscillator makes a small phase adjustment in each cycle and synchronizes the clock to an exact 24-hour period (photoentrainment). WT mice and the single Opn4^{-/-} and rd/rd mutants entrained normally and consolidated their wheel-running activity to the dark period of the LD cycle (FIG. 4). In contrast, the Opn4^{-/-} rd/rd mice failed to entrain to the external lighting cycle, and continued to exhibit free-running rhythms (FIG. 4, Table 1). In addition, increasing the light intensity to 800 lux during the photoperiod and increasing the photoperiod to 12 hr failed to entrain these mice (FIG. 4, FIG. 5, and Table 1).

TABLE 1
Period length estimates and acute suppression of activity by light. Average period length
estimates or percent activity suppression + SEM (number of animals) are shown. Average values
significantly different from that of the WT (Students t-test, two tailed, equal variance, p < 0.005) are
in bold letters. Corresponding p-values are shown beneath the averages.

| | WT | Opn4^{-/-} | rd/rd | Opn4^{-/-}; rd/rd |
|---|---|---|---|---|
| Period length (hr) | | | | |
| 8 hr 100lux: 16 hr dark | 24.00 + 0.003 (8) | 23.99 + 0.015 (15) | 24.02 + 0.009 (9) | 23.44 + 0.087 (5) |
| | | 0.1603 | 0.3541 | 2.96E−14 |
| 8 hr 800lux: 16 hr dark | 24.00 + 0.01 (17) | 23.97 + 0.01 (8) | 23.98 + 0.02 (16) | 23.49 + 0.06 (11) |
| | | 0.0466 | 0.2451 | 2.2E−11 |
| 12 hr 800lux: 12 hr dark | 24.00 + 0.01 (7) | 23.97 + 0.03 (3) | 24.00 + 0.003 (13) | 23.52 + 0.06 (3) |
| | | 0.1523 | 0.5974 | 1.62E−06 |
| Constant darkness | 23.55 + 0.1 (5) | 23.64 + 0.06 (7) | 23.6 + 0.05 (7) | 23.28 + 0.06 (4) |
| | | 0.6037 | 0.4062 | 0.0587 |
| Constant light (100lux) | 25.28 + 0.14 (17) | 24.33 + 0.23 (6) | 25.65 + 0.27 (7) | 23.32 + 0.16 (8) |
| | | 0.001 | 0.092 | 6.73E−09 |
| Light suppression of activity (%) | | | | |
| 2 hr 100lux | 75.00 + 3.16 (10) | 38.91 + 9.79 (5) | 56.24 + 7.82 (11) | 2.35 + 9.79 (5) |
| | | 0.0033 | 0.0838 | 2.34E−05 |
| 2 hr 800lux | 93.25 + 3.87 (15) | 64.95 + 13.40 (6) | 90.45 + 4.85 (10) | 0.98 + 2.56 (8) |
| | | 0.0023 | 0.2795 | 1.7884E−14 |

All four genotypes exhibited free-running DD periods of less than 24 hours (Table 1). Under constant light (LL) conditions, most nocturnal rodents demonstrate lengthening of their free-running periods. In LL, WT and rd/rd mice free ran with comparable period of more than 24 hrs. Opn4^{-/-} mice exhibited a slightly shorter (albeit >24 hr) period length in LL. The Opn4^{-/-}; rd/rd mice, however, continued to free run with an unchanged, <24 hr period length (Table 1, FIG. 5), comparable to the period of free-run in DD.

The entrainment phenotype of Opn4^{-/-}; rd/rd mice is thus comparable to that of bilaterally enucleated mice, which also free run in LD conditions (Nelson & Zucker, Comparative Biochemical Physiology 69A: 145-148, 1981), suggesting that the entrainment deficiency in these mice is a result of complete loss of photic input to a functioning oscillator in the SCN. Loss of photic entrainment has also been reported in math5^{-/-} (Wee, et al., J Neurosci 22:10427-33, 2002), a mutant that fails to develop most retinal ganglion cells, and in anophthalmic mice (Faradji, et al., Brain Res 202:41-9, 1980). However, the intrinsic period length of the oscillator in these mice is lengthened. We suspect that the Opn4^{-/-}; rd/rd mouse does not mimic these severe developmental mutants due to the presence of an intact optic nerve and, presumably, retinohypothalamic tract. Most likely a light-independent interaction between the inner retina and the SCN neurons is necessary to finely determine the free-running period of the SCN oscillator.

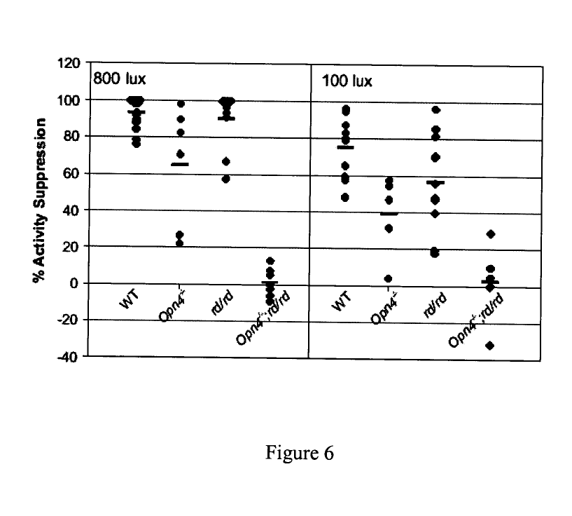

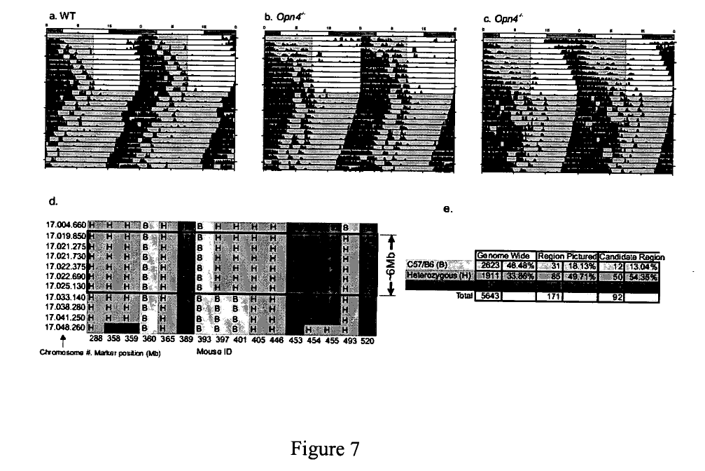

Acute suppression of activity (masking) was tested by a briefpulse of light during the dark phase of the LD cycle (7). Opn4^{-/-} mice showed reduced masking responses to a 2 hr pulse of white light (100 lux or 800 lux) administered during the first half of the dark phase (FIG. 6). However, variability was observed in this response; masking was found to be further reduced in presence of an unlinked genetic locus (FIG. 7). Rd/rd littermate animals also exhibited reduced masking under low irradiance. In contrast, Opn4^{-/-}; rd/rd showed no masking responses under any irradiance conditions tested (FIG. 6, Table 1). As Opn4^{-/-} and rd/rd mice each exhibit partial deficiency in masking under these lighting conditions, complete absence of masking in the double mutant Opn4^{-/-}; rd/rd mice demonstrates the partially redundant role of these light signaling pathways.

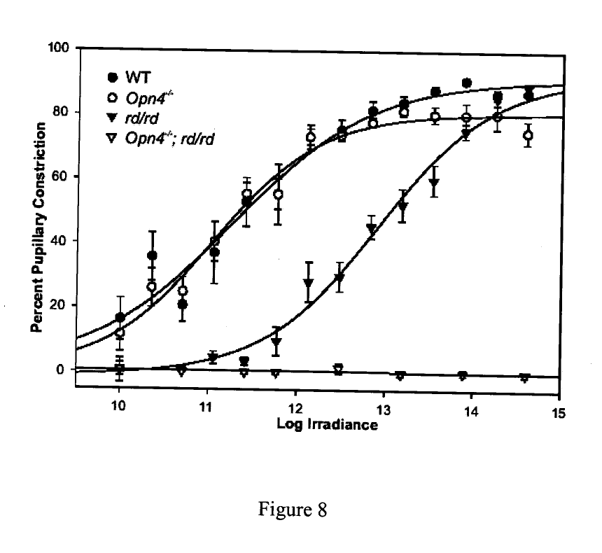

The PLR to 470 nm blue light was compared among WT, Opn4^{-/-}, rd/rd, and Opn4^{-/-}; rd/rd mice. The PLR of Opn4^{-/-} and WT littermate control mice were comparable, although at high irradiance levels the maximal pupillary constriction of Opn4^{-/-} mice was less than WT. While rd/rd mice showed a ˜1 log unit decrease in sensitivity compared with WT animals, Opn4^{-/-}; rd/rd mice showed no pupillary constriction at any intensity tested (FIG. 8). These mice showed normal pupillary constriction following topical application of pilocarpine, suggesting no defect in pupillary motor function. Thus, melanopsin is absolutely required for PLR in rd/rd mice.

The synthesis of the pineal hormone melatonin is acutely suppressed by light (Klein and Weller, Science 177532-3, 1972). Photic suppression of melatonin also persists in the absence of rods and cones (Lucas, et al., Science 284:505-7, 1999), suggesting a possible role of melanopsin-expressing ipRGCs in this photoresponse. Arylalkylamine N-acetyltransferase (AA-NAT, E.C. 2.3.1.87) is the rate-limiting enzyme of the melatonin biosynthetic pathway. The nocturnal rise in AA-NAT mRNA is acutely inhibited by light (Roseboom, et al., Endocrinology 137:3033-45, 1996). Photoinhibition of AA-NAT mRNA was measured with quantitative RT-PCR as follows.

Wheel running activity was monitored in mice maintained in an 8L:16D photoperiod. Experimental groups were exposed to extended light until 9 h after the predicted time of activity onset (equivalent to 9 h after dark onset in those genotypes that are photoentrained). Animals were anesthetized with isoflurane, and pineals were excised and immediately placed in 10 μl of RNAlater (Ambion Inc., Austin, Tex.). Total RNA was extracted (RNAqueous™-Micro; Ambion Inc.) and reverse transcribed (Superscript™ II Reverse Transcriptase Preamplification System; Invitrogen, Carlsbad, Calif.). Relative quantities of AA-NAT mRNA were determined in a ABI PRISM GeneAmp 5700 Sequence Detection System (Applied Biosystems Inc., Foster City, Calif.) using SYBR Green and AA-NAT specific primers (forward, 5′-CAG CCC CCA GGA CAA CAC-3′; reverse, 5′-GGT TCC CCA GCT TCA GAA GTG-3′) that span the first intron and were designed using Primer Express 1.5 software (Applied Biosystems Inc) according to the GenBank sequence accession number NM_{—}009591.1. The presence of a single amplimer of appropriate size was confirmed by melting curve analysis.

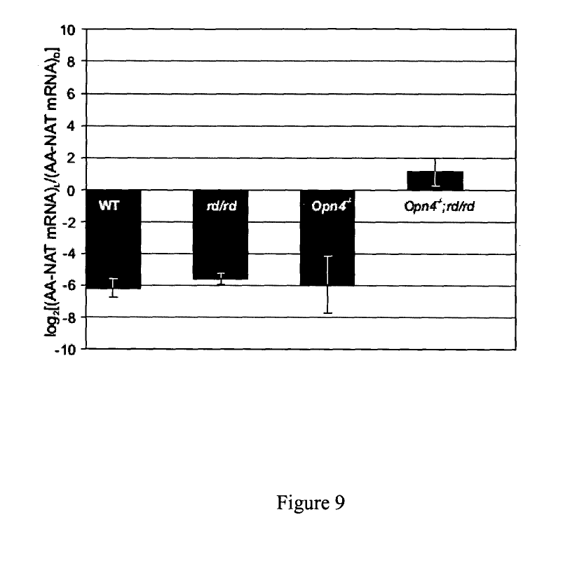

WT, rd/rd, and Opn4^{-/-} mice demonstrated photic inhibition of AA-NAT mRNA transcription. In contrast, Opn4^{-/-}; rd/rd mice showed no photic inhibition of AA-NAT transcription (FIG. 9).

The most parsimonious explanation for the severe deficiency in non-visual photic responses in the Opn4^{-/-}; rd/rd mice is that either the melanopsin-containing ipRGCs or the classical outer retinal photoreceptors are sufficient for transducing photic information to critical brain areas. At least partial functional redundancy, thus, exists between rods and/or cones and melanopsin-containing ipRGCs for non-visual photoreception. Whether the classical photoreceptors function by signaling “through” the ipRGCs (via synaptic input to these cells) is not known, but is suggested by the synaptic contacts of bipolar and amacrine cells onto melanopsin-expressing ipRGCs (Belenky, et al., J Comp Neurol 460:380-93, 2003). Differences in the neurocircuitry and downstream signaling pathways may underlie the observed differences in the relative contributions of classical rod and cone photoreceptors and of melanopsin containing ipRGCs to non-visual photophysiology.

The complete loss of photic responses in Opn4^{-/-}; rd/rd mice also demonstrates that no additional photopigments are required for non-visual photic signaling. Cryptochromes, which function as circadian photopigments in Arabidopsis (Somers, et al., Science 282:1488-90, 1998) and Drosophila (Emery, et al., Cell 95:669-79, 1998), are also expressed in the mammalian eye (Miyamoto and Sancar, Proc Natl Acad Sci USA 95:6097-102, 1998). However, a subset of rd/rd mice lacking cryptochromes still show masking responses (Van Gelder, et al., J Neurogenet 16:181-203, 2002), and pupillary responses are intact under very bright light (Van Gelder et al., Science 299:222, 2003). Melanopsin appears to be expressed normally in eyes of cryptochrome-deficient mice (Van Gelder, et al., J Neurogenet 16:181-203, 2002). Thus, the primary photopigment in non-visual photoreception is melanopsin-dependent, but not cryptochrome-dependent.

1. A transgenic knockout mouse whose genome comprises a disruption in the mouse's endogenous melanopsin gene, wherein the disruption prevents the expression of a functional melanopsin protein in cells of the mouse.

2. The transgenic knockout mouse of claim 1, wherein the mouse comprises a homozygous disruption of the melanopsin gene.

3. The method of claim 1, the homozygous disruption results in the transgenic knockout mouse exhibiting an attenuated circadian rhythm phase-shift in response to a light pulse during a dark portion of an environmental dark/light cycle.

4. A cell isolated from the transgenic knockout mouse of claim 1, wherein the genome of the cell comprises a disruption in its endogenous melanopsin gene, and wherein the homozygous disruption prevents the expression of a functional melanopsin protein in said cell.

5. A method for identifying a therapeutic agent for modulating circadian rhythm in a mammal, the method comprising:
administering an agent to a transgenic knockout animal whose genome comprises a disruption in its endogenous melanopsin gene, wherein the disruption prevents the expression of a functional melanopsin protein in cells of the animal and the animal comprises a homozygous disruption of the melanopsin gene; and
selecting an agent that modulates the regulation of circadian rhythm in the animal.

6. The method of claim 5, wherein the knockout animal displays an attenuated circadian rhythm phase-shift response to a light pulse during a dark portion of an environmental dark/light cycle.

7. The method of claim 5, wherein the selecting step comprises selecting an agent that enhances the animal's circadian rhythm phase-shift response to a light pulse during a dark portion of an environmental dark/light cycle.

8. The method of claim 5, wherein the animal is a mouse.

9. A method of modulating circadian rhythm in a mammal in need thereof, the method comprising administering to the mammal an effective amount of the agent selected in claim 5.

10. The method of claim 9, wherein timing of administration of the selected agent is pre-determined to coincide with an appropriate phase of an existing circadian rhythm to produce a selected modulation of the circadian rhythm.

11. The method of claim 9, wherein the selected agent is used to treat or prevent a sleep disorder.

12. The method of claim 9, wherein the mammal has a condition selected from the group selected from insomnia, Seasonal Affective Disorder, Shift Work dysrhythmia, delayed-sleep phase syndrome, and jet-lag.

13. The method of claim 9, wherein the mammal is a human.

14. The method of claim 9, wherein the selected agent is administered in conjunction with melatonin or a compound that suppresses or stimulates melatonin production.

15. The method of claim 9, wherein the selected agent is administered in conjunction with light therapy.

16. A method of modulating circadian rhythm in a mammal in need thereof, the method comprising administering to the mammal an effective amount of a melanopsin modulator.

17. The method of claim 16, wherein timing of administration of the modulator is pre-determined to coincide with an appropriate phase of an existing circadian rhythm to produce a selected modulation of the circadian rhythm.

18. The method of claim 16, wherein the modulator is used to treat or prevent a sleep disorder.

19. The method of claim 16, wherein the mammal has a condition selected from the group selected from insomnia, Seasonal Affective Disorder, Shift Work dysrhythmia, delayed-sleep phase syndrome, and jet-lag.

20. The method of claim 16, wherein the mammal is a human.

21. The method of claim 16, wherein the modulator is administered in conjunction with melatonin or a compound that suppresses or stimulates melatonin production.


SEQUENCE LISTING (12 sequences)

SEQ ID NO: 1 - DNA, 2137 bp, Mus sp.
  mouse melanopsin cDNA
cactcattcctttgcgcttcattggacattaagcagtcagcagcccaaagagcagctccaggctggatggatgagagcgggcagcaggtggaccaggccgcagggttaaggatggtatagagccggaagtctggggaccgatccctgatctttccatggccttagctcctctgagagcctgagcatggactctccttcaggaccaagagtcttgtcaagcttaactcaggatcccagcttcacaaccagtcctgccctgcaaggcatttggaacggcactcagaacgtctccgtaagagcccagcttctctctgttagccccacgacatctgcacatcaggctgctgcctgggtccccttccccacagtcgatgtcccagaccatgctcactataccctaggcacggtgatcctgctggtgggactcacagggatgctgggcaatctgacggtcatctacaccttctgcaggaacagaggcctgcggacaccagcaaacatgttcatcatcaacctcgcagtcagcgacttcctcatgtcagtcactcaggccccggtcttctttgccagcagcctctacaagaagtggctctttggggagacaggttgcgagttctatgccttctgcggggctgtctttggcatcacttccatgatcaccctgacagccatagccatggaccgctatctggtgatcacacgtccactggccaccatcggcaggggatccaaaagacgaacggcactcgtcctgctaggcgtctggctttatgccctggcctggagtctgccacctttctttggttggagtgcctacgtgcccgaggggctgctgacatcctgctcctgggactacatgaccttcacaccccaggtgcgtgcctacaccatgctgctcttctgctttgtcttcttcctccccctgctcatcatcatcttctgctacatcttcatcttcagggccatccgagagacaggccgggcctgtgagggctgcggtgagtcccctctgcggcagaggcggcagtggcagcggctgcagagtgagtggaagatggccaaggtcgcactgattgtcattcttctcttcgtgctgtcctgggctccctactccactgtggctctggtggcctttgctggatactcgcacatcctgacgccctacatgagctcggtgccagccgtcatcgccaaggcttctgccatccacaatcccattatctacgccatcactcaccccaagtacagggtggccattgcccagcacctgccttgccttggggtgcttctcggtgtatcaggccagcgcagccacccctccctcagctaccgctctacccaccgctccacattgagcagccagtcctcagacctcagctggatctctggacggaagcgtcaagagtccctgggttctgagagtgaagtgggctggacagacacagaaacaaccgctgcatggggagctgcccagcaagcaagtggacagtccttctgcagtcagaacctagaagatggagaactcaaggcctcttccagcccccaggtacagagatctaagactcccaaggtgcctggacccagtacctgccgccctatgaaaggacagggagccaggccaagtagcctaaggggtgaccagaaaggcaggcttgctgtgtgcacaggcctctcagagtgtccccatccccatacatcccagtttccccttgctttcctagaggatgatgtgactctcagacatctgtagcagggtctaagtatgatctgtatctaggggaatatctgcatgtgactgtgtagctctgcgcatgacatgctgtcagctatgttgtaccatatgtatatgtagagtatgcatataacttatgtgcccttgaagatatgtggcctacagcagagaacaactcatgcgtgtgtggaccatgttcctggcatatatgctctctgtcactgtgatgcctctgtgttgtgtgggtgacagagtgtgatggtgttcacctctctgcgcgggttttgatgctgggcaaacacggggaagggagctgcaagccatgtactagctcactgccgatggcctgtgctcaagatgtcaccgaggagaacacttgtagctattaaaagaaggccagctgtc

SEQ ID NO: 2 - protein, 521 aa, Mus sp.
  mouse melanopsin
MDSPSGPRVLSSLTQDPSFTTSPALQGIWNGTQNVSVRAQLLSVSPTTSAHQAAAWVPFPTVDVPDHAHYTLGTVILLVGLTGMLGNLTVIYTFCRNRGLRTPANMFIINLAVSDFLMSVTQAPVFFASSLYKKWLFGETGCEFYAFCGAVFGITSMITLTAIAMDRYLVITRPLATIGRGSKRRTALVLLGVWLYALAWSLPPFFGWSAYVPEGLLTSCSWDYMTFTPQVRAYTMLLFCFVFFLPLLIIIFCYIFIFRAIRETGRACEGCGESPLRQRRQWQRLQSEWKMAKVALIVILLFVLSWAPYSTVALVAFAGYSHILTPYMSSVPAVIAKASAIHNPIIYAITHPKYRVAIAQHLPCLGVLLGVSGQRSHPSLSYRSTHRSTLSSQSSDLSWISGRKRQESLGSESEVGWTDTETTAAWGAAQQASGQSFCSQNLEDGELKASSSPQVQRSKTPKVPGPSTCRPMKGQGARPSSLRGDQKGRLAVCTGLSECPHPHTSQFPLAFLEDDVTLRHL

SEQ ID NO: 3 - DNA, 2284 bp, Homo sapiens
  human melanopsin cDNA
cggacacaggagaaagcagcgggtaggctaagcaggggtgctgaggatggaggaaagttgggaggctgagcacagctgaagtcctgagctccctgtgcccttgacttctctgtgggctcgagcaaggaccatcccaactcaggatgaaccctccttcggggccaagagtcccgcccagcccaacccaagagcccagctgcatggccaccccagcaccacccagctggtgggacagctcccagagcagcatctccagcctgggccggcttccatccatcagtcccacagcacctgggacttgggctgctgcctgggtccccctccccacggttgatgttccagaccatgcccactataccctgggcacagtgatcttgctggtgggactcacggggatgctgggcaacctgacggtcatctataccttctgcaggagcagaagcctccggacacctgccaacatgttcattatcaacctcgcggtcagcgacttcctcatgtccttcacccaggcccctgtcttcttcaccagtagcctctataagcagtggctctttggggagacaggctgcgagttctatgccttctgtggagctctctttggcatttcctccatgatcaccctgacggccatcgccctggaccgctacctggtaatcacacgcccgctggccacctttggtgtggcgtccaagaggcgtgcggcatttgtcctgctgggcgtttggctctatgccctggcctggagtctgccacccttcttcggctggagcgcctacgtgcccgaggggttgctgacatcctgctcctgggactacatgagcttcacgccggccgtgcgtgcctacaccatgcttctctgctgcttcgtgttcttcctccctctgcttatcatcatctactgctacatcttcatcttcagggccatccgggagacaggacgggctctccagaccttcggggcctgcaagggcaatggcgagtccctgtggcagcggcagcggctgcagagcgagtgcaagatggccaagatcatgctgctggtcatcctcctcttcgtgctctcctgggctccctattccgctgtggccctggtggcctttgctgggtacgcacacgtcctgacaccctacatgagctcggtgccagccgtcatcgccaaggcctctgcaatccacaaccccatcatttacgccatcacccaccccaagtacagggtggccattgcccagcacctgccctgcctgggggtgctgctgggtgtatcacgccggcacagtcgcccctaccccagctaccgctccacccaccgctccacgctgaccagccacacctccaacctcagctggatctccatacggaggcgccaggagtccctgggctcggagagtgaggtgggctggacacacatggaggcagcagctgtgtggggagctgcccagcaagcaaatgggcggtccctctacggtcagggtctggaggacttggaagccaaggcaccccccagaccccagggacacgaagcagagactccagggaagaccaaggggctgatccccagccaggaccccaggatgtaggacgcccactggctctccctttcttctgagacacatccagcccccccacgtctccctcatatacacagacccaggattatgctgtgagcctgcaggctttggaagtggccctgtcacccgtgctgcacgggattcacagccccagccccatggcccctctccacacctcaaaactcctgccccataacgtcctccgcatccactttccagctcagcagccgcacccgaggctcagcctgaggggtgtgtgcccaggccctcccacttcccgagttgtctgcctctcctcaaatgctgtgtgctgcaattgtccaggcgatgacaatggtgatggctccagagaacacaccagctatttatgagcctctgcccccaggctgggcctgtcactggcataggaaggccagccccgcatctcccactgccaacagctgaagccgagcacagacctccctttgcacgctggaacagttactcacctgtggcttcttcccccagtgtaccgttccactgtggcccacattcttgtgcacgcgggcatttgcaggcacgctctcgcgtagttacctatctgaatgcacaccaagcacatgcgtgcacactctgcgtctgtgattcatttcatgtagtggtctaagctcctcccagggctgtgtggatctgacagggtataggaaaataaaaagcggagaaggtgtcttcag

SEQ ID NO: 4 - protein, 478 aa, Homo sapiens
  human melanopsin
MNPPSGPRVPPSPTQEPSCMATPAPPSWWDSSQSSISSLGRLPSISPTAPGTWAAAWVPLPTVDVPDHAHYTLGTVILLVGLTGMLGNLTVIYTFCRSRSLRTPANMFIINLAVSDFLMSFTQAPVFFTSSLYKQWLFGETGCEFYAFCGALFGISSMITLTAIALDRYLVITRPLATFGVASKRRAAFVLLGVWLYALAWSLPPFFGWSAYVPEGLLTSCSWDYMSFTPAVRAYTMLLCCFVFFLPLLIIIYCYIFIFRAIRETGRALQTFGACKGNGESLWQRQRLQSECKMAKIMLLVILLFVLSWAPYSAVALVAFAGYAHVLTPYMSSVPAVIAKASAIHNPIIYAITHPKYRVAIAQHLPCLGVLLGVSRRHSRPYPSYRSTHRSTLTSHTSNLSWISIRRRQESLGSESEVGWTHMEAAAVWGAAQQANGRSLYGQGLEDLEAKAPPRPQGHEAETPGKTKGLIPSQDPRM

SEQ ID NO: 5 - DNA, 25 bp, Artificial Sequence
  Description of Artificial SequencePrimer a
caggagcaaggtgagatgacaggag

SEQ ID NO: 6 - DNA, 25 bp, Artificial Sequence
  Description of Artificial SequencePrimer b
aggatggtatagagccggaagtctg

SEQ ID NO: 7 - DNA, 25 bp, Artificial Sequence
  Description of Artificial SequencePrimer c
tcaagccacagaggatactagcagg

SEQ ID NO: 8 - DNA, 25 bp, Artificial Sequence
  Description of Artificial SequencePrimer d
gatgatctggacgaagagcatcagg

SEQ ID NO: 9 - DNA, 25 bp, Artificial Sequence
  Description of Artificial SequencePrimer e
actgaggactgacactgaagcctgg

SEQ ID NO: 10 - DNA, 25 bp, Artificial Sequence
  Description of Artificial SequencePrimer f
cagtgtcaggcctagcgggaagaga

SEQ ID NO: 11 - DNA, 18 bp, Artificial Sequence
  Description of Artificial Sequencequantitative RT-PCR arylalkylamine N-acetyltransferase (AA-NAT) specific forward primer
cagcccccaggacaacac

SEQ ID NO: 12 - DNA, 21 bp, Artificial Sequence
  Description of Artificial Sequencequantitative RT-PCR arylalkylamine N-acetyltransferase (AA-NAT) specific reverse primer
ggttccccagcttcagaagtg